(19) United States
(12) Patent Application Publication
(10) Pub. No.: US 2016/0051536 A1
(43) Pub. Date: Feb. 25, 2016

(54) COMPOSITIONS TO ALLEVIATE PRESYSTEMIC METABOLISM OF OPIOIDS

(71) Applicants: VIRGINIA COMMONWEALTH UNIVERSITY, Richmond, VA (US); INDIVIOR UK LIMITED, Berkshire (GB)

(72) Inventors: Phillip M. GERK, Richmond, VA (US); Azmi NASSER, Richmond, VA (US)

(73) Assignee: Virginia Commonwealth University, Richmond, VA (US)

(21) Appl. No.: 14/781,683

(22) PCT Filed: Apr. 8, 2014
(86) PCT No.: PCT/US2014/033290

§ 371 Date: Oct. 1, 2015

Related U.S. Application Data
(60) Provisional application No. 61/809,669, filed on Apr. 8, 2013.

(51) Int. Cl. A61K 31/485; A61K 31/085; A61K 31/235; A61K 31/7048; A61K 31/357; A61K 31/352; A61K 31/05; A61K 31/11; A61K 31/12

(52) U.S. Cl. CPC A61K 31/485; A61K 31/11; A61K 31/085; A61K 31/235; A61K 31/12; A61K 31/357; A61K 31/352; A61K 31/05; A61K 31/7048

(57) ABSTRACT

Compositions comprising one or more opioids and one or more inhibitors of uridine diphosphate glucuronosyl transferases (UGTs) are provided. The inhibitors decrease the presystemic metabolism of the one or more opioids, thereby increasing their bioavailability. The inhibitors are compounds that are designated as Generally Regarded as Safe (GRAS) and/or “Everything Added to Food” (EAF) and/or are dietary supplements. Methods of alleviating pain and of treating opiate addiction in a subject by administering the compositions are also provided.

### BACKGROUND

1. Field of the Invention

The invention is related to increasing the bioavailability of active agents such as opioids. In particular, the active agents are administered in conjunction with various Generally Regarded as Safe (GRAS) and/or “Everything Added to Food” (EAF) compounds and/or dietary supplements which inhibit glucuronidation, thereby decreasing presystemic metabolism of the agent and increasing bioavailability.

2. Description of the Prior Art

Increasing the bioavailablity of compounds provided to a subject to treat various diseases has been a subject of intense investigation for a number of years, with drugs that are used for the treatment of pain being of special importance. Among these, opioids are among the world's oldest known and frequently prescribed drugs for managing pain; their therapeutic use predates recorded history. The analgesic (painkilling) effects of opioids are due to decreased perception of pain, decreased reaction to pain as well as increased pain tolerance.

Unfortunately, opioid addiction and abuse is also a serious health problem in the US and throughout the world. Treatment of addiction has met with varying degrees of success, and several strategies have been used. For example, buprenorphine is 100-fold more potent than morphine, but unlike morphine, it is a partial μ-opiate receptor antagonist and thus has also been used in the treatment of addiction. However, drug abusers may crush, dissolve and inject tablets intended for sublingual use, thus abusing buprenorphine itself. In order to prevent this behavior, buprenorphine and the opioid antagonist naloxone are used together (e.g. Suboxone™). This sublingual formulation is usually dosed ranging from 2:0.5 mg to 12:3 mg buprenorphine:naloxone. Unfortunately, buprenorphine and naloxone both have low oral bioavailability and extensive presystemic metabolism in the intestine and liver. Buprenorphine is metabolized by CYP3A4-mediated N-dealkylation to form norbuprenorphine. Buprenorphine, norbuprenorphine and naloxone also undergo glucuronidation and their glucuronide metabolites are excreted into bile and are thought to undergo enterohepatic recirculation. While the sublingual dosage form is intended to escape intestinal and hepatic presystemic metabolism, thereby delivering a dose that will be sufficient to alleviate symptoms of addiction and decrease motivation for abusing the composition, without inducing toxicity, the net bioavailability of sublingual buprenorphine is in the range of only 28-51%, and is insufficient to be effective in many cases. Further, a fraction of the administered dose is still swallowed, and this fraction has extremely low bioavailability.

There is a need in the art for safely improving the bioavailability of opioids and other bioactives.

U.S. Pat. No. 5,972,382 to Majeed et al. teaches compositions and methods for the improvement of gastrointestinal absorption and systemic utilization of nutrients and nutritional supplements by combining them with piperine, an alkaloid derived from black pepper. Majeed does not discuss the delivery of drugs per se, and piperine is not a GRAS compound.

U.S. Pat. No. 7,576,124 to Harris describes “first-pass” inhibiting furocoumarin compounds that are purportedly safe and effective. The furocoumarins are citrus-derived substances prepared from, e.g., grapefruit. Harris does not identify which components of pre-systemic metabolism are inhibited, but the cytochrome P450 family of enzymes is referenced. The furocoumarins are not described as GRAS.

U.S. Pat. No. 7,125,564 to Chen et al. discusses problems associated with first-pass degradation of bioactive treatment compounds, and teaches the use of water-soluble complexes with glycyrrhizin, which is the main sweet-tasting compounds from licorice root. Glycyrrhizin is described as GRAS. Chen does not indicate that glycyrrhizin can inhibit first pass metabolism; rather, Chen discusses having the compositions parenterally administered to avoid the first-pass effect.

U.S. Pat. No. 7,070,814 to Qazi et al. teaches compositions which are purportedly bioenhancing/bioavailability-facilitating. These compositions include an extract and/or at least one bioactive fraction from the Cuminum cyminum plant (i.e., the plant from which the spice cumin is derived). This extract is combined with drugs, nutrients, vitamins, nutraceuticals, herbal drugs/products, micro nutrients, and antioxidants, along with pharmaceutically acceptable additives/excipients. Similar to the Majeed patent, Qazi discusses optionally including piperine (or extract/fraction of piper nigrum or piper longum) to purportedly increase the beneficial effect of the extract. Qazi is particularly focused on the problem of pre-systemic metabolism of drugs and suggests that the compositions described in the patent may function by inhibiting or reducing the rate of biotransformation of drugs in the liver or intestines. Qazi does not identify the extract as including GRAS compounds.

U.S. Pat. No. 6,180,666 to Wacher et al. describes orally co-administering a compound of interest with a gallic acid ester such as octyl gallate, propyl gallate, lauryl gallate, and methyl gallate. Gallic acid is a trihydroxybenzoic acid, a type of organic phenolic acid found in plants such as gallnuts, sumac, witch hazel, tea leaves, and oak bark. The gallic acid ester is purportedly present in order to inhibit biotransformations of drugs that are carried out e.g. by cytochromes P450. The esters are described as GRAS compounds. However, Wacher does not describe particular synergistic combinations of UGT inhibitors to increase opioid bioavailability.

U.S. Pat. No. 6,121,234 to Benet et al., describes a method for purportedly increasing bioavailabity and reducing inter- and intra-individual variability of an orally administered hydrophobic pharmaceutical compound. In Benet, the pharmaceutical compound is orally co-administered with an essential oil or essential oil component. Benet suggests that the role of the essential oil may be to inhibit drug biotransformation in the gut. Essential oils are described as GRAS compounds.

US patent application 2003/0215462 to Wacher et al. describes using UDP-glucuronosyltrasnsferase (UGT) inhibitors to increase the bioavailability orally administered drugs. Wacher suggests the formulation may be used with 2-methoxyestradiol, raloxifene, irinotecan, SN-38, estradiol, labetalol, dilevalol, zidovudine (AZT) and morphine. The UDP-inhibitors are generally natural products and include epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin, and silymarin. Wacker does not list buprenorphine and naloxone as exemplary drugs, nor are the GRAS substances propyl paraben, vanillin, vitamin C and curcumin identified as being useful in Wacher. The objective of the Wacher technology appears to be the identification of specific combinations of drugs and inhibitors that work well together. Further, Wacher does not describe particular synergistic combinations of UGT inhibitors to increase opioid bioavailability.

US patent applications 2006/0040875 and 2009/0074708 to Oliver et al. describe UGT2B inhibitors that can increase the bio-availability of drugs. Specifically named inhibitors are natural products such as capillarisin, isorhamnetin, β-naphthoflavone, α-naphthoflavone, hesperetin, terpineol, (+)-limonene, β-myrcene, swertiamarin, eriodictyol, cineole, apigenin, baicalin, ursolic acid, isovitexin, lauryl alcohol, puerarin, trans-cinnamaldehyde, 3-phenylpropyl acetate, isoliquritigenin, paeoniflorin, gallic acid, genistein, glycyrrhizin, protocatechuic acid, ethyl myristate, and umbelliferone. Suggested drugs for which bioavailability can be increased include morphine, naloxone, nalorphine, oxymorphone, hydromorphone, dihydromorphine, codeine, naltrexone, naltrindole, nalbuphine and buprenorphine. The focus of Oliver is on the delivery of analgesics. However, Oliver does not describe particular synergistic combinations of UGT inhibitors to increase opioid bioavailability.

US patent application 2010/0087493 to Kaivosaari et al. teaches a method for increasing bioavailability of a pharmacologically active agent that undergoes direct N-glucuronidation by UDP-glucuronosyltransferase isoenzyme UGT2B10 by administering an UGT2B10 modulator, e.g. an inhibitor of UGT2B10 (preferably selectively for UGT2B10 over UGT1A4). The drugs for which bioavailability may be increased are described as having a nucleophilic nitrogen atom, including primary, secondary and tertiary aryl- and alkylamines, sulfonamides and aromatic or aliphatic heterocyclic compounds having one or more nitrogen atoms as heteroatoms. Nicotine is identified as an example. The inhibitors are not described in detail, and only Levomedetomidine is provided as an example. In addition, Kaivosaari does not describe particular synergistic combinations of UGT inhibitors to increase opioid bioavailability.

WO/2011/026112 describes methods of increase bioavailabity of a pharmaceutically active agent by using specific inhibitors of a UGT that glucuronidates the pharmaceutically active agent. However, in WO/2011/026112, the inhibitors are described as comprising an N-acyl phenylaminoalcohol residue and a uridine moiety connected by a spacer. Thus, the use of GRAS compounds is not described in WO/2011/026112.

WO/2010/015636 teaches beta-carbolin-derivatives to inhibit metabolic enzymes and thereby increase bioavailability of drugs such as antibiotics. However, the use of GRAS compounds or the application to opioids is not discussed.

WO/2013/049365, the complete contents of which is hereby incorporated by reference in entirety, discloses the use of dietary and GRAS compounds to increase the bioavailability of selected compounds, but opioids are not discussed.

It would be advantageous to have available additional compositions which include GRAS compounds and bioactive agents such as opioids.

### SUMMARY

The invention provides novel combinations of compounds which comprise at least one bioactive (e.g. an opioid) and one or more inhibitors of an enzyme or enzymes that catalyze unwanted presystemic metabolism of the bioactive(s) by UGTs, thus allowing the bioactive(s) to be absorbed intact, and increasing bioavailability. Practice of the invention advantageously results in lower dosage formulations, since less of a given active agent must to used to achieve the same therapeutic effect (e.g. analgesia) effect. Toxicity of the drug formulations is thus minimized. The inhibitors used in the practice of the invention are generally chosen from the Federal Drug Administration's (FDA's) list of GRAS compounds and/or other dietary substances which are known or believed to be safe for consumption. The inhibitor may also have an “Everything Added to Food” (EAF) designation, i.e. there is reported use of the substance, but it has not yet been assigned for toxicology literature search by the FDA. In some aspects, particular combinations of opioids and inhibitors are provided, e.g. combinations that display synergistic inhibitory effects.

The novel combinations of the invention may be used in any of many ways in which opioids are employed. Exemplary uses include but are not limited to the treatment of pain, and the treatment of addiction, for which combinations of buprenorphine, naloxone and one or more inhibitors of glucuronidation are particularly advantageous.

The invention provides methods of providing one or more opioids to a subject in need thereof so as to enhance systemic bioavailiability of the one or more opioids. The methods comprise the step of: providing the one or more opioids to the subject in combination with one or more inhibitors of one or more uridine diphosphate glucuronosyl transferases (UGTs), wherein the one or more inhibitors of one or more UGTs are classified as Generally Regarded as Safe (GRAS), “Everything Added to Food” (EAF) and/or as a dietary supplement. In one aspect, the one or more opioids includes buprenorphine. The one or more opioids may include buprenorphine and naloxone. In some aspects, the one or more inhibitors of one or more UGTs inhibits at least one of UGT1A1, UGT1A3, UGT1A4, UGT1A5, UGT1A6, UGT1A7, UGT1A8, UGT1A9, UGT1A10, UGT2A1, UGT2A2, UGT2A3, UGT2B4, UGT2B7, UGT2B10, UGT2B11, UGT2B15, UGT2B17 and UGT2B28. The methods may further comprise a step of providing said subject with one or both of: i) one or more inhibitors of at least one cytochrome P450 monooxygenase (CYP) selected from the group consisting of CYP1A1, CYP1A2, CYP2A6, CYP2D6, CYP2C9, CYP2C8, CYP2C18, CYP2C19, CYP3A4, CYP3A5 and CYP3A7; and ii) one or more inhibitors of at least one sulfotransferase (SULT) selected from the group consisting of SULT1A1, SULT1A2, SULT1A3, SULT1C4 and SULT2B1. In yet other aspects, the one or more inhibitors are selected from the group consisting of propylparaben, ethyl vanillin, eugenol, vanillin, quercetin, resveratrol, isoeugenol, methylparaben, zingerone, piperine, ethyl vanillin propylene, glycol acetal, curcumin, pterostilbene, propylgallate, rasketone, magnolol, guaiacol, α-mangostin, silybin, and pinoresinol. The one or more inhibitors may be administered as a combination of inhibitors selected from the group consisting of: isoeugenol and propyl gallate; vanillin and isoeugenol; and vanillin, isoeugenol and propyl gallate. In some aspects, the step of providing is performed orally or intra-rectally, intra-vaginally, or intra-urethrally.

The invention also provides dosage form of one or more opioids, comprising: one or more opioids and one or more UGT inhibitors that is/are classified as Generally Regarded as Safe (GRAS), “Everything Added to Food” (EAF) and/or as a dietary supplement. In one aspect, the one or more opioids includes buprenorphine. The one or more opioids may include buprenorphine and naloxone. In some aspects, the one or more inhibitors of one or more UGTs inhibits at least one of UGT1A1, UGT1A3, UGT1A4, UGT1A5, UGT1A6, UGT1A7, UGT1A8, UGT1A9, UGT1A10, UGT2A1, UGT2A2, UGT2A3, UGT2B4, UGT2B7, UGT2B10, UGT2B11, UGT2B15, UGT2B17 and UGT2B28. The methods may further comprise a step of providing said subject with one or both of: i) one or more inhibitors of at least one cytochrome P450 monooxygenase (CYP) selected from the group consisting of CYP1A1, CYP1A2, CYP2A6, CYP2D6, CYP2C9, CYP2C8, CYP2C18, CYP2C19, CYP3A4, CYP3A5 and CYP3A7; and ii) one or more inhibitors of at least one sulfotransferase (SULT) selected from the group consisting of SULT1A1, SULT1A2, SULT1A3, SULT1C4 and SULT2B1. In yet other aspects, the one or more inhibitors are selected from the group consisting of propylparaben, ethyl vanillin, eugenol, vanillin, quercetin, resveratrol, isoeugenol, methylparaben, zingerone, piperine, ethyl vanillin propylene, glycol acetal, curcumin, pterostilbene, propylgallate, rasketone, magnolol, guaiacol, a-mangostin, silybin, and pinoresinol. The one or more inhibitors may be administered as a combination of inhibitors selected from the group consisting of: isoeugenol and propyl gallate; vanillin and isoeugenol; and vanillin, isoeugenol and propyl gallate. The dosage form may be formulated for oral, rectal, vaginal or urethral administration.

The invention further provides methods of treating or preventing pain in a subject in need thereof, the method comprising a step of administering to said subject a composition comprising one or more opioids and one or more UGT inhibitors that is classified as Generally Regarded as Safe (GRAS), “Everything Added to Food” (EAF) and/or as a dietary supplement. The one or more opioids may include buprenorphine. In some aspects, the one or more UGT inhibitors is a combination of inhibitors selected from the group consisting of: isoeugenol and propyl gallate; vanillin and isoeugenol; and vanillin, isoeugenol and propyl gallate.

In further aspects of the invention, methods of treating or preventing opiate dependency or addiction in a subject in need thereof are provided. The methods comprise a step of administering to the subject a composition comprising buprenorphine and naloxone; and one or more UGT inhibitors that is classified as Generally Regarded as Safe (GRAS), “Everything Added to Food” (EAF) and/or as a dietary supplement. The one or more UGT inhibitors may be selected from the group consisting of eugenol, isoeugenol, ethyl vanillin, vanillin, curcumin, silybin A, α-mangostin, resveratrol, propyl gallate, and naringin, and may include e.g. 2, 3, 4, 5, 6, 7, 8, 9 or 10 these inhibitors.

For any of the above methods or dosage forms, the one or more inhibitors may be a combination of at least two inhibitors selected from the group consisting of eugenol, isoeugenol, ethyl vanillin, vanillin, curcumin, silybin A, α-mangostin, resveratrol, propyl gallate, and naringin.

For any of the above methods or dosage forms, the one or more UGT inhibitors may include a combination of alkylated catechols and other phenolic compounds.

### DESCRIPTION OF THE DRAWINGS

FIG. 1. Structures of exemplary opioids.

FIG. 2. Structural category, Federal Drug Administration (FDA) status and typical dose ranges for exemplary inhibitors.

FIG. 3. Time course of buprenorphine-glucuronide formation in LS 180 cells.

FIGS. 4A and B. A and B, effect of various inhibitors on buprenorphine-glucuronide formation in LS 180 cells.

FIGS. 5A and B. Effect of various inhibitors (A) and inhibitor combinations (B) on buprenorphine-glucuronide formation in LS 180 cells.

FIGS. 6A and B. Effect of various inhibitors on buprenorphine-glucuronide formation in LS 180 cells in A, graphic and B, tabular form.

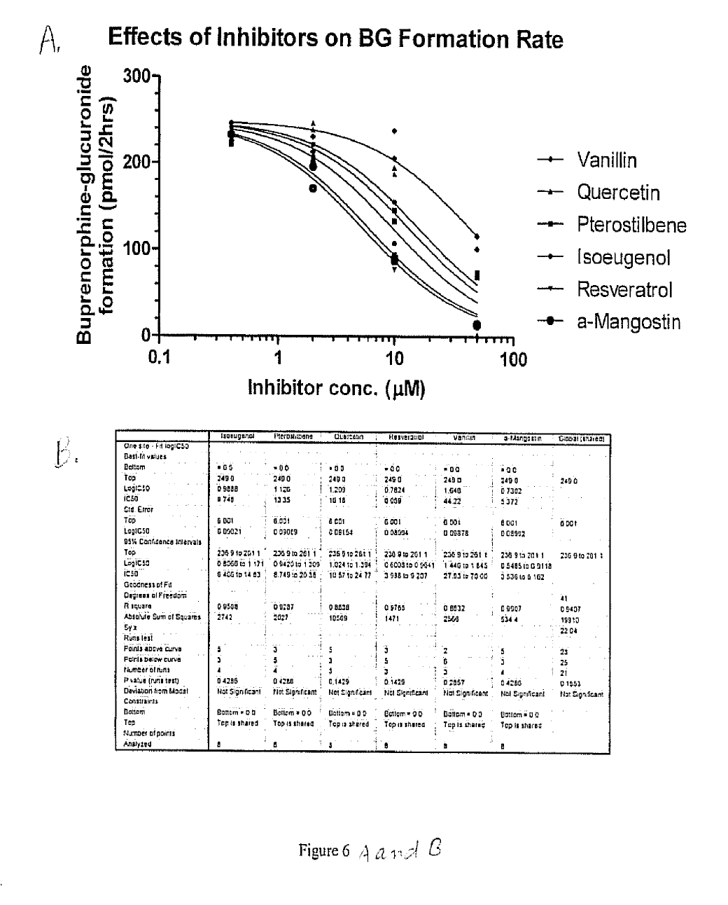

FIG. 7. Plot of the IC₅₀ values for the inhibitors isoeugenol, pterostilbene, quercetin, resveratrol, vanillin, and α-mangostin vs their calculated c Log P values.

FIG. 8. Buprenorpine levels in serum of rats with and without inhibitors.

### DETAILED DESCRIPTION

Combinations of at least one active agent (e.g. one or more opioids) and one or more inhibitors of enzymes that catalyze unwanted presystemic metabolism of the active agent(s) (e.g. uridine diphosphate glucuronyltransferases [glucuronosyltransferases, UGTs], and, optionally, cytochrome P450 monooxygenases [CYPs], and sulfotransferases [SULTs]) are provided, as is their use for bringing about a biological effect in a subject in need thereof, e.g. alleviating pain (inducing analgesia), or other beneficial or desired therapeutic effect. The inclusion of the inhibitors in the compositions described herein advantageously results in higher levels (concentrations) of biologically available active agent (e.g. an opioid) in the circulatory system of the patient. In addition, use of the inhibitors in the compositions also advantageously permits lower amounts of an active agent to be administered, while achieving the same effect (e.g. analgesia), thereby decreasing unwanted side effects. Generally, the increase in the level of active agent is in the range of from at least about 2 fold to about 100 fold or more, e.g. at least about a 2, 3, 4, 5, 6, 7, 8, 9, or 10 fold increase is achieved, or even a 20, 25, 30, 35, 40, 45, 50, 55, 60, 65, 70, 75, 80, 85, 90 or 95-fold increase or more may be achieved. In some aspects, the increase is 20, 25 or 50 fold. A corresponding decrease in the amount of active agent that is needed in a formulation to achieve a desired effect is in the range of from at least about 2 fold to about 100 fold or more (with exemplary decreasing fold amounts as listed above for increases). This advantage could be especially helpful when treating vulnerable populations such as the elderly, weakened terminally ill patients, patients receiving other medications, etc.

As used herein, an “opioid” is any psychoactive chemical that resembles morphine or other opiates in its pharmacological effects. Opioids work by binding to opioid receptors, which are found principally in the central and peripheral nervous system, but also in the gastrointestinal and respiratory tracts. The receptors in these organ systems mediate both the beneficial effects and the side effects of opioids. Although the term opiate is often used as a synonym for opioid, the term opiate is properly limited to the natural alkaloids found in the resin of the opium poppy (Papaver somniferum), while opioid refers to both opiates and synthetic substances, as well as to opioid peptides.

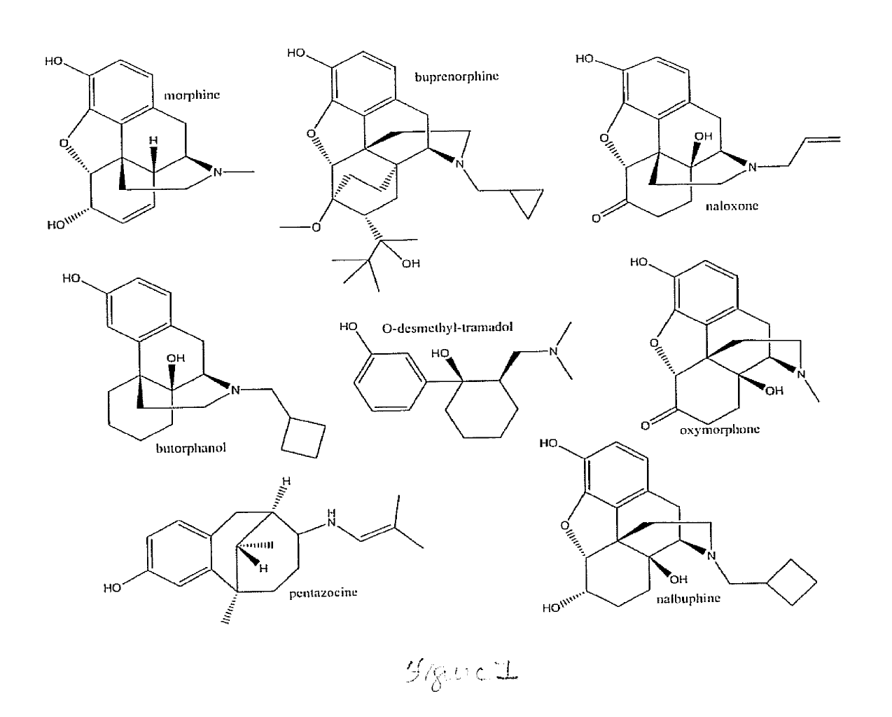

Opioids for which bioavailability may be increased by co-administration with one or more dietary and/or GRAS compounds as described herein include but are not limited to: buprenorphine, naloxone, morphine, meperidine, hydromorphone, oxymorphone, naltrexone, nalmefene, levorphanol, butorphanol, pentazocine, nalbuphine, levallorphan, ketobemidone, dezocine, tapentadol, meptazinol, methylnaltrexone, O-desmethyl-tramadol, norbuprenorphine, tramadol, codeine, etorphine, etc. Exemplary opioids are depicted in FIG. 1.

The opioid compositions described herein may be administered to lessen or alleviate pain. Exemplary uses of the opioid compositions described herein include but are not limited to: treatment of acute pain (such as post-operative pain); in palliative care to alleviate severe, chronic, disabling pain; in terminal conditions such as cancer; for degenerative conditions such as rheumatoid arthritis; for the pain caused by fibromyalgia or migraine; for chronic neuropathic pain; to relieve labor pain during childbirth; to manage dyspnea (shortness of breath) particularly in advanced diseases such as advanced cancer, and the like. Opioids for pain relief are also used when nondrug pain treatment options including cognitive behavioral therapy, exercise, spinal manipulation, and physical medicine and rehabilitation programs are insufficient to meet therapy goals. All such conditions or symptoms of illness or disease may be treated in a patient in need thereof by administering a therapeutically effective dose of one or more of the compositions disclosed herein. The present invention encompasses methods of treating an individual or subject in need of such therapy. The invention also provides methods of administering one or more opioids to a subject using the compositions provided herein. The methods may involve a step of identifying a patient/subject who is in need of administration or who could benefit from administration of the active agent(s) that is/are in the formulations, followed by a step of administering or providing the formulation to the subject, and may also include one or more steps of monitoring the results of administration, e.g. the progress of the patient.

In addition, some combinations of one or more opioids plus one or more inhibitors are used to treat patients suffering from and/or recovering from opiate (narcotic) dependence or addiction. The invention provides method of treating opiate addiction. “Addiction” refers to compulsive drug use despite harmful consequences and is characterized by an inability to stop using a drug, even when objectively it is in the individual's best interest to do so, attendant with other psycho-social side effects, e.g. failure to meet work, social, or family obligations; and, sometimes (depending on the drug), tolerance and withdrawal symptoms when the drug is not available. “Dependence” refers to physical dependence in which the body adapts to the drug, requiring more of it to achieve a certain effect (tolerance) and eliciting drug-specific physical or mental symptoms if drug use is abruptly ceased (withdrawal). Thus, physical dependence in and of itself does not constitute addiction, but it often accompanies addiction. The use and/or abuse of opiates such as morphine, heroin, codeine, oxycodone, hydrocodone, oxymorphone, fentanyl, and others can result in dependence and/or addiction. Symptoms of opiate dependence, addiction and withdrawal can be prevented and/or treated (e.g. ameliorated, lessened, decreased, etc.) by administration of the combinations of e.g. buprenorphine, naloxone and one or more enzyme inhibitors as described herein. Various biological, psychological and/or social impairment, and various combinations and interactions thereof, may be alleviated by administration of the compositions described herein, e.g. buprenorphine plus naloxone plus one or more enzyme inhibitors. Symptoms that are alleviated include but are not limited to: a strong desire or sense of compulsion to take the drug; difficulties in controlling drug-taking behavior in terms of its onset, termination, or levels of use; a physiological withdrawal state when drug use is stopped or reduced, as evidenced by: the characteristic withdrawal syndrome for the substance; or use of the same (or a closely related) substance with the intention of relieving or avoiding withdrawal symptoms; evidence of tolerance, such that increased doses of the drug are required in order to achieve effects originally produced by lower doses; progressive neglect of alternative pleasures or interests because of drug use, increased amount of time necessary to obtain or take the drug or to recover from its effects; persisting with drug use despite clear evidence of overtly harmful consequences, such as harm to the liver, depressive mood states or impairment of cognitive functioning, etc.

The methods may involve a step of identifying a patient/subject who is in need of administration or who could benefit from administration of the active agent(s) that is/are in the formulations, followed by a step of administering a therapeutically effective dose of the active agents to the subject. The method may also include one or more steps of monitoring the results of administration, e.g. the progress of the patient.

The opioid that is administered as described herein (e.g. buprenorphine) may be provided in a unit dosage form of from about 0.1 mg to about 100 mg, e.g. from about 2 mg to about 100 mg, or from about 0.5 mg to about 50 mg, with exemplary doses being e.g. about 2, 4, 8, 12, 16, or 24 mg. In one aspect, the invention provides a formulation comprising buprenorphine plus naloxone plus one or more enzyme inhibitors. Generally, in compositions comprising both buprenorphine and naloxone, the ratio of buprenorphine:naloxone is at most about 1:1, and is generally about 2:1, 2.5:1, 3:1, 3.5:1, or about 4:1, or even 4.5:1 or 5:1, in terms of mg of active agent. Naloxone is generally present in the range of from about 0.5 mg to about 25 mg per dose. Additional information regarding suitable doses and dose ranges for buprenorphine and/or buprenorphine plus naloxone are described in issued U.S. Pat. No.: 8,658,198 (Petterson); U.S. Pat. No. 8,652,529 (Guimberteau); U.S. Pat. No. 8,652,515 (Sackler); U.S. Pat. No. 8,637,540 (Kumar), U.S. Pat. No. 8,481,560 (Stinchcomb), and U.S. Pat. No. 7,964,610 (Lewis) and U.S. Pat. No. 7,402,591 (Chapleo), and US patent application 2003/0191147 (Sherman), the complete contents of each of which is hereby incorporated by reference in entirety, as are the references cited therein. A dosage form contains an appropriate amount of drug to provide a therapeutic effect, i.e. is a therapeutically effective dose.

In addition, the opiate that is administered may be in the form of a pharmaceutically acceptable salt. “Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenarnine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The opioid (e.g. buprenorphine) may also be administered as a prodrug, or as another physiologically acceptable derivative or modified form, e.g. as an ester. Further, various biologically/therapeutically effective analogs, homologues, and polymorphs thereof, as well as mixtures of any of the foregoing, may be utilized.

In some aspects, the active agents are administered as a formulation comprising more than one active, e.g. 2, 3, 4, or 5 or more active agents may be administered in a single formulation. For example, the opioids buprenorphine and naloxone may be combined together in a single oral dosage form which also includes at least one enzyme inhibitor as described herein, e.g. at least one UGT inhibitor, and, optionally, at least one CYP inhibitor and/or at least one SULT inhibitor. In some aspects, the at least one UGT inhibitor is a combination of UGT inhibitors that interact synergistically. Exemplary synergistic combinations include but are not limited to: isoeugenol and propyl gallate; vanillin and isoeugenol; and vanillin, isoeugenol and propyl gallate.

In one aspect, the invention provides methods of treating opiate addiction by administering to a subject in need thereof a combination of buprenorphine plus naloxone plus one or more inhibitors as described herein. A typical treatment regimen, involves, for example, identification of a suitable subject, followed by initial “day one” administration of 4 mg of buprenorphine as buprenorphine:naloxone 3 times,in order to load the patient's system with a substantial amount of the drugs, and observation of the patient's response. On day 2, one dose is typically administered (e.g. up to about 16 mg, depending on the attending medical professional's assessment of the results of the initial dosing). Further observation of the effect on the subject is taken into consideration. On day 3 and thereafter, the dose is adjusted to an amount that stabilizes the patient and that is efficacious, e.g. one dose per day that is sufficient to decrease or eliminate the subject's desire or craving for the opiate to which he/she is addicted. Treatment typically is carried out using the final, stabilized dose for about 1-2 weeks, or longer as necessary, until the subject is deemed to have been withdrawn from opioid dependence, and can safely discontinue therapy without relapse.

A variety of GRAS and/or EAFUS and/or dietary compounds may be used as enzyme inhibitors in the present invention, including but not limited to: ascorbic acid, niacin, vanillin, ethyl vanillin, vanillin, quercetin, resveratrol, isoeugenol, methylparaben, zingerone, piperine, ethyl vanillin propylene glycol acetal, curcumin, pterostilbene, propylgallate, rasketone (raspberry ketone), magnolol, guaiacol, α-mangostin, silybin, pinoresinol, propylparaben, eugenol, propyl gallate, zingerone, naringin, cinnamic acid, sinapic acid, caffeic acid, ferrulic acid, cinnamaldehyde, kuromanin (cyanidin 3-O-glucoside), etc.

Inhibitor compounds of particular interest include phenols and/or alkylated catechols. Exemplary phenols include but are not limited to: propylparaben, quercetin, resveratrol, methylparaben, pterostilbene, propyl gallate, raspberry ketone (a.k.a. rasketone), and magnolol, etc. Exemplary alkylated catechols include but are not limited to: ethyl vanillin, eugenol, vanillin, isoeugenol, zingerone, piperine, ethyl vanillin propylene glycol acetal, curcumin, guaiacol, α-mangostin, silybin, and pinoresinol, etc.

Additional compounds and combinations of compounds which may be employed to increase the bioavailability of orally provided bioactives include but are not limited to: methyl paraben, ethyl paraben, propyl paraben, butyl paraben, (−)-homoeriodictyol; 2,6-dimethoxyphenol; 2-i sopropylphenol; 2-methoxy-4-methylphenol; 2-methoxy-4-propylphenol; 4-(1,1-dimethylethy phenol; 4-allylphenol; 4-ethylguaiacol; 4-ethylphenol; anisyl alcohol; butylated hydroxyanisole; butylated hydroxytoluene; carvacrol; carveol; dimethoxybenzene; divanillin; essential oils and extracts (e.g., clove, cinnamon, nutmeg, rosemary, citrus, vanilla, ginger, guaiac, turmeric, grape seed, black pepper, etc.); ethyl p-anisate; eugenyl acetate; eugenyl formate; isoeugenol and various forms thereof (including but not limited toacetate, formate, or benzoate-isoeugenol); L-tyrosine; methyl anisate; methylphenyl ether; methylphenyl sulfide; O-(ethoxymethyl)phenol; O-cresol; O-propylphenol; resorcinol; salicylates (amyl, benzyl, butyl, ethyl, methyl, etc.); thymol; trans-anethole; vanillin propylene glycol acetal; vanillyl acetate; vanillyl alcohol; vanillyl ethyl ether; vanillylidene acetone; veratraldelhyde; and xylenols (2,6-; 2,5-; 3,4-).

Additional herbal/natural compounds not on GRAS/EAFUS list which may be used to increase the bioavailability of orally provided bioactives include hesperetin; eriodictyonone; 5,3′-dihydroxy-7,4′-dimethoxyflavanone; isorhamnetol; tamarixetin; syringetin; 3′, 7-dimethylquercetin; and methylated and/or dehydroxylated analogs of quercetin.

Additional compounds which may be used include flavonoids which include but are not limited to: flavanols (such as catechin, gallocatechin, epicatechin, catechin gallate, gallocatechin gallate, epigallocatechin, epicatechin gallate, epigallocatechin gallate, leucoanthocyanidin, and proanthocyanidins), flavones (such as luteolin, apigenin, tangeretin), flavonols (such as quercetin, kaempferol, myricetin, fisetin, isorhamnetin, pachypodol, rhamnazin), flavanones (such as hesperetin, hesperidin, eriodictyol, homoeriodictyol), flavanonols (such as taxifolin, dihydroquerectin, dihydrokaempferol), anthocyanidins (such as anthocyanidin, cyanidin, delphidin, malvidin, pelargonidin, peonidin, petunidin), isoflavones (such as genistein, daidzein, glycitein), isoflavanes (such as equol, lonchocarpane, laxiflorane), and neoflavonoids (such as dalbergin, nivetin, coutareagenin, dalbergichromene). Glycosides of the flavanols, flavonol, flavones, flavanones, flavanonols, anthocyanidins, isoflavones, isoflavanes, and neoflavonoids may also be used. Flavonolignans (such as silybin, silybinin A, silybin B, silydianin, silychristin, isosilychristin, isosilybin A, isosilybin B, silibinin, silychristin, silydianin, dehydrosilybin, deoxysilycistin, deoxysilydianin, silandrin, silybinome, silyhermin and neosilyhermin, silyamandin, hydnocai pin, scutellaprostin A, B, C, D, E and F; hydnowightin, palstatin, salcolin A and salcolin B, rhodiolin) and their glycosides may also be used in the practice of the invention. Lignans (pinoresinol, steganacin, enterodiol, enterolactone, lariciresinol, secoisolariciresinol, matairesinol, hydroxymatairesinol, syringaresinol and sesamin) and their glycosides would be included. Xanthones (alpha-mangostin, beta-mangostin, gamma-mangostin, garcinone, garcinone A, garcinone C, garcinone D, mangostanol, gartanin) and their glycosides may be used in the practice of the invention. Miscellaneous natural phenolic compounds may also be included such as hydroxy-methoxy-coumarins, hydroxy-chalcones, biochanin A, prunetin, kavalactones (1,1-hydroxyyangonin; 1,1-methoxy-12-hydroxydehydrokavain; 5-hydroxykavain), ellagic acid, rosmarinic acid, emodin, and amentoflavone.

Other inhibitory compounds which may be used in the practice of the invention include but are not limited to GRAS/EAFUS compounds such as: (−)-homoeriodictyol (EAFUS-EAF); 2,6-dimethoxyphenol; 2-isopropylphenol; 2-methoxy-4-methylphenol; 2-methoxy-4-propylphenol; 4-(1,1-dimethylethyl)phenol; 4-allylphenol; 4-ethylguaiacol; 4-ethylphenol; anisyl alcohol; butylated hydroxyanisole; butylated hydroxytoluene; carvacrol; carveol; dimethoxybenzene; divanillin (EAFUS-EAF); essential oils and extracts (e.g., clove, cinnamon, nutmeg, rosemary, citrus, vanilla, ginger, guaiac, turmeric, grape seed, black pepper, etc.); ethyl p-anisate; eugenyl acetate; eugenyl formate; isoeugenol (acetate, formate, or benzoate); L-tyrosine; methyl anisate; methylphenyl ether; methylphenyl sulfide; O-(ethoxymethyl)phenol; O-cresol; O-propylphenol; resorcinol; salicylates (amyl, benzyl, butyl, ethyl, methyl, etc.); thymol; trans-anethole; vanillin propylene glycol acetal; vanillyl acetate; vanillyl alcohol; vanillyl ethyl ether; vanillylidene acetone; veratraldelhyde; and xylenols (2,6-; 2,5-; 3,4-).

Combinations of inhibitor compounds may also be employed. Exemplary combinations include but are not limited to: propylparaben and ascorbic acid; propylparaben and vanillin; eugenol and propylparaben; eugenol and vanillin; eugenol, propylparaben, vanillin, and ascorbic acid; curcumin and resveratrol; curcumin, pterostilbene, resveratrol, and zingerone; pterostilbene and zingerone; two or more of vanillin, isoeugenol and propyl gallate (e.g. vanillin and isoeugenol; vanillin and propyl gallate; isoeugenol and propyl gallate; or vanillin, isoeugenol and propyl gallate), as well as eugenol, isoeugenol, ethyl vanillin, vanillin, curcumin, silybin A, α-mangostin, resveratrol, propyl gallate, and naringin; two or more of resveratrol, curcumin, quercetin, isoeugenol, and propyl gallate. These exemplary combinations may be used, for example, in formulations for the treatment of drug addiction which also include buprenorphine and naloxone, as described elsewhere herein.

In some aspects, a combination of inhibitors is used and the effect of the co-administration of more than one inhibitor results in (displays) synergy (is synergistic). By “synergy” we mean the joint action of agents is such that when taken together, they increase each other's effectiveness, i.e. synergy refers to the working together of two or more agents (e.g. drugs, inhibitors, etc.) to produce an effect greater than the sum (e.g. arithmetic sum) of their individual effects. Exemplary synergistic combinations of inhibitors that are used in the practice of the present invention include but are not limited to: isoeugenol and propyl gallate; vanillin & isoeugenol; vanillin, isoeugenol and propyl gallate.

Furthermore, suitable inhibitory compounds and combinations of compounds for use in the practice of the invention can be readily identified using enzymatic activity assays such as those disclosed in WO/2013/049365.

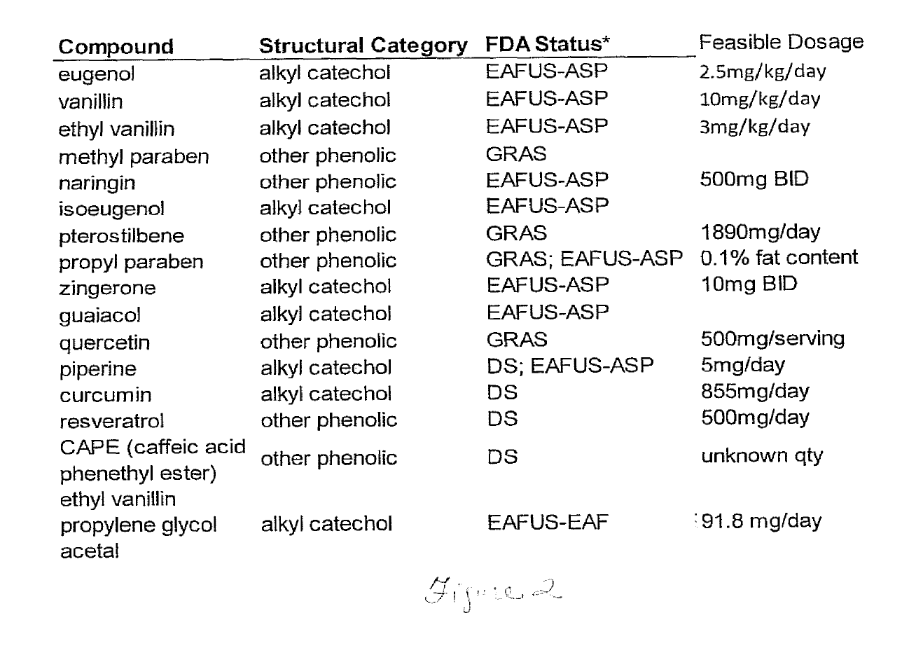

The dose of enzyme inhibitor that is administered to a patient may vary according to e.g. the amount that is necessary to effectively inhibit the enzyme of interest, e.g. at least one of a UGT, a CYP and/or an SULT. In general, the dose will range from at least about 0.1 mg/kg/day to about 40 mg/kg/day (or more), and may be e.g. about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/kg/day. The dose of enzyme inhibitor that is administered to a patient may vary according to e.g. the amount that is necessary to effectively inhibit the enzyme of interest, e.g. at least one of a UGT, a CYP and/or an SULT. In general, the dose will range from about 0.1 mg/kg per day to about 30 mg/kg/day, and may be e.g. 2 mg/kg/day. Dosing of the active compounds (i.e., opioids) and the inhibitors may occur with a frequency between once weekly to six times per day, and may be e.g. once daily. In terms of daily dosage, for an average 70 kg patient, one may expect dosing to be as follows: vanillin, about 50 to 1000 mg/day, e.g. about 200 mg/day; eugenol, about 50 to 250 mg/day, e.g. about 150 mg/day; ethyl vanillin, about 50 to about 300 mg/day, e.g. about 200 mg/day; naringin, about 50 to about 1500 mg/day, e.g. about 500 mg/day; isoeugenol, about 50 to 250 mg/day, e.g. about 150 mg/day; pterostilbene, about 150 to about 2500 mg/day, e.g. about 1500 mg/day; propylparaben, about 50 to about 150 mg/day, e.g. about 100 mg/day; zingerone, about 10 to 30 mg/day, e.g. about 20 mg/day; quercetin, about 50 to about 2000 mg/day, e.g. about 1000 mg/day; curcumin, about 50 to about 1500 mg/day, e.g. about 800 mg/day; resveratrol, about 100 to about 750 mg/day, e.g. about 500 mg/day; ethyl vanillin propylene glycol acetal, about 50 to about 150 mg/day, e.g. about 92 mg/day; raspberry ketone (rasketone), about 50 to 200, e.g. about 100 mg/day; magnolol, about 50 to 200, e.g. about 100 mg/day; honokiol, about 50 to 200 mg/day, e.g. about 100 mg/day; guaiacol, about 20 to 100, e.g. about 50 mg/day; a-mangostin, about 500 to 1500 mg, e.g. about 1000 mg/day; silybin (silymarin), about 100 to 1000, e.g. about 420 mg/day; pinoresinol, about 10 to 500 mg/day, e.g. about 100 mg/day; propyl gallate, about 14 to 140 mg/day, e.g. about 70 mg/day. Exemplary doses of representative inhibitors are presented in FIG. 2. Doses may be administered, e.g. once daily or more often e.g. 2, 3, 4, or even more times per day, with individual doses being adjusted so that the total for the day is generally within the recommended exemplary ranges.

Exemplary combinations of inhibitors include but are not limited to:
i) isoeugenol and propyl gallate;
ii) vanillin and isoeugenol;
iii) vanillin, isoeugenol and propyl gallate; and
iv) eugenol, isoeugenol, ethyl vanillin, vanillin, curcumin, silybin A, α-mangostin, resveratrol, propyl gallate, and naringin. For this exemplary combination of 10 inhibitors, the formulation may include 2, 3, 4, 5, 6, 7, 8, 9 or all 10 of the inhibitors.

Exemplary enzymes that are inhibited as described herein include but are not limited to:

UGTs: UGT isoforms UGT1A1, UGT1A3, UGT1A4, UGT1A5, UGT1A6, UGT1A7, UGT1A8, UGT1A9, UGT1A10, UGT2A1, UGT2A2, UGT2A3, UGT2B4, UGT2B7, UGT2B10, UGT2B11, UGT2B15, UGT2B17, UGT2B28,

CYPs: CYP isoforms CYP1A1, CYP1A2, CYP2A6, CYP2D6, CYPC9, CYP2C8, CYP2C18, CYP2C19, CYP3A4, CYP3A5, CYP3A7, CYP2J2, CYP4A1, CYP2E1, CYP2B6

SULTs: SULT isoforms SULT 1A1, SULT 1A2, SULT 1A3, SULT 1B1, SULT1C4, SULT 2A1, SULT 2B1, etc.

The inhibition of one or more of the above enzymes does not preclude the inhibition of other presystemic metabolic enzymes by the inhibitors of the compositions, as an inhibitor may inhibit more than one enzyme, e.g. more than one of the above, or more than one other enzyme.

The present invention provides compositions (formulations) comprising one or more opioids and one or more enzyme inhibitors as described herein (e.g. a UGT inhibitor), and a pharmacologically suitable carrier. Each ingredient in the composition is essentially or substantially pure, prior to incorporation into the composition. Typically, such compositions may be prepared as liquid solutions or suspensions, or as solid forms such as tablets, pills, powders, wafers (e.g. that dissolve when held on or under the tongue for sublingual administration), as suppositories, emulsions, microemulsions, nanoemulsions, and self-emulsifying (self-microemulsifying, self-nano-emulsifying) dosage forms., and the like. Further, solid forms suitable for solution in, or suspension in, liquids prior to administration may also be prepared. The preparations may also be emulsified. The formulations may include excipients which are pharmaceutically acceptable and compatible with the active ingredients. Suitable excipients are, for example, solvents or co-solvents, including but not limited to: water, saline, dextrose, glycerol, ethanol, N-methyl-2-pyrrolidone, dimethylacetamide, propylene glycol, polyethylene glycols of various molecular weights (e.g., 300, 400, 600, 1000, 3350, 4000), castor oil and derivatives, peppermint oil, vegetable oils (e.g. peanut, corn, olive, safflower, sesame, soybean, coconut, palm oils) and the like, or combinations thereof. In addition, the composition may contain minor amounts of auxiliary substances such as wetting, suspending agents (cellulose derivatives, including hydroxypropylcellulose, methylcellulose, carboxymethylcellulose, various cyclodextrins, and the like), or emulsifying agents (such as polysorbates, including polysorbate 80 (TWEEN® 80), polysorbate 20 (TWEEN® 20), sorbitan esters (Spans), polyvinyl alcohol, polyvinylpyrrolidone, oleic acid, D-α-tocopheryl polyethylene glycol 1000 succinate (TPGS), poloxamer 407, as well as various proprietary detergents, such as LABRASOL®, GELUCIRE ®, CREMAPHOR®, BRIJ®, SOLUTOLS®, LABRAFILS®, SOFTIGENS®, and their variants; pH buffering agents, and the like. For orally administered forms of the composition, various thickeners, flavorings, diluents, emulsifiers, dispersing aids, binders, components related to slow release of the active agent(s), and the like may be added. The composition of the present invention may contain any such additional ingredients so as to provide the composition in a form suitable for administration. The compositions may be formulated as a food product, e.g. into wafers or “candy”, treats, etc. that are taken orally; and/or may be formulated with the intention of administration by adding them to a food product, e.g. a powder or crystals to be added a flavored drink, to drinking water, to a nutrition booster, etc. The compositions may be formulated for rapid or slow release. The final amount of active agent(s) in the formulations may vary. However, in general, the amount in the formulations will be from about 0.1-99%. The final amount of inhibitor(s) in the formulations may vary. However, in general, the amount in the formulations will be from about 0.1-99%. Still other suitable formulations for use in the present invention can be found, for example in Remington's Pharmaceutical Sciences, Philadelphia, Pa., 19th ed. (1995).

A goal of the present invention is to avoid presystemic metabolism and/or degradation of the active agent(s) that is/are administered. Thus, administration is generally oral, e.g. by mouth via swallowing and/or chewing or dissolving (in the oral cavity) of a composition, which may be a liquid or solid. However, any method of administration which results in exposure of the active agent to presystemic metabolizing enzymes may be used.

In addition, the compositions may be administered in conjunction with other treatment modalities such as substances that boost the immune system, various chemotherapeutic agents, anti-cancer agents, antibiotic agents, and the like. The additional treatment modalities may be administered in compositions separate from those which contain the opioid(s) plus inhibitor(s), but administered in coordination therewith, or may be included in the same composition.

Subjects to whom the compositions of the invention are administered are generally animals, usually mammals, and may be humans (e.g. adults and elderly men and women. However, the invention also encompasses veterinary applications of the technology, e.g. for companion pets (dogs, cats, ferrets, hamsters, etc), live stock (cattle, pigs, goats, sheep, etc.), other commercially valuable non-human mammals (horses, etc.), as well as animals in protected areas, e.g. zoos, preserves, etc. The present compositions are ideal for some of these applications since they are designed for oral administration, and can thus be included, e.g. in a food product for the animal. The non-human animal may be a juvenile or adult.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Before exemplary aspects of the present invention are described in greater detail in the Examples below, it is to be understood that this invention is not limited to particular aspects described, as such may, of course, vary. It is also to be understood that the terminology used herein is for the purpose of describing particular aspects only, and is not intended to be limiting, since the scope of the present invention will be limited only by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

### EXAMPLES

Unless otherwise indicated, the in vitro experiments described in the following section were conducted using human Caucasian colon adenocarcinoma LS180 cells. These cells are recognized in the art as a human intestinal cell culture model. They express a wide range of drug-metabolizing enzymes, similar to those found in the small intestine. They grow readily and provide reproducible results. LS180 cells also express ATP-binding cassette (ABC) transporters (P-glycoprotein, multidrug resistance-associated proteins (MRPs), etc.).

### Example 1

### Effects of Inhibitors on the Formation of Buprenorphine-Glucuronide in LS 180 Cells

Phenolic opioids typically have low (e.g. <50%) oral bioavailability. In addition, the bioavailability is highly variable from patient to patient. This variability is largely due to extensive presystemic metabolism such as glucuronidation, although oxidation and some sulfation are also observed.

Human intestinal cells (LS180) were cultured in Dulbecco's modified Eagle's medium (DMEM) supplemented with 10% fetal bovine serum and 1% non-essential amino acids in microplates. Solutions of buprenorphine (10 μM) were prepared in Dulbecco's phosphate buffered saline (DPBS), in the presence or absence of inhibitors, as listed (100 μM). The cells were rinsed with DPBS, aspirated, and the solutions (37° C.) were added to the cells and incubated for 2 hours. The reactions were stopped and protein was precipitated by adding 20% acetonitrile. After freezing (−80° C.) and thawing, samples were centrifuged and analyzed by HPLC using an Alltima HP C18 column (4.6×100 mm, 3 μm) with gradient elution (mobile phase A: acetonitrile; mobile phase B: 10% acetonitrile, 90% aqueous 25 mM ammonium acetate; 0-78% A) followed by detection by fluoresence (excitation 283 nm, emission 346 nm) and quantitation using Waters Empower 2 software. Results were analyzed by one-way ANOVA with Dunnett's post-test (control vs. inhibitors); asterisk designates p<0.05; n.d. designates not detected.

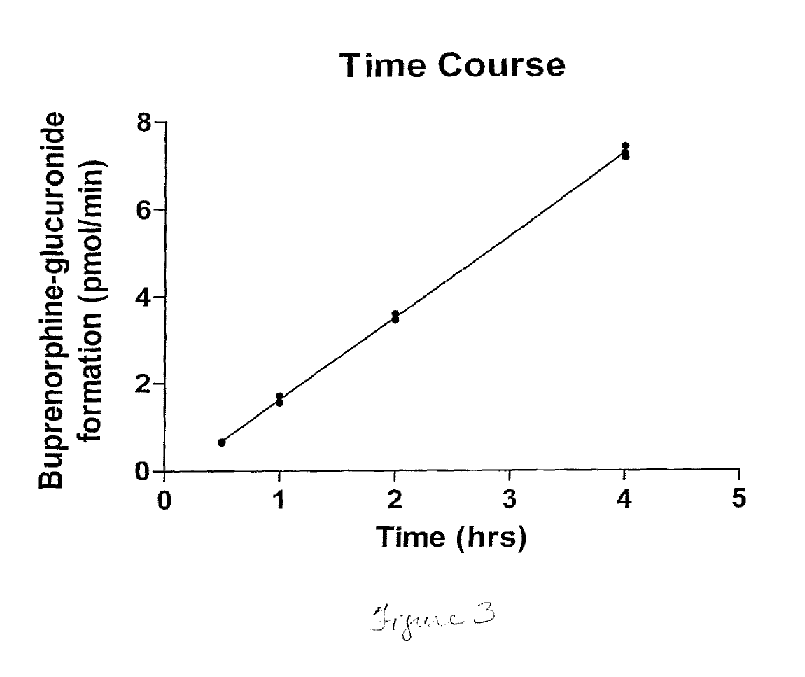

FIG. 3 shows the in vitro (in LS 180 cells) time course of the formation of buprenorphine-glucuronide, the inactive metabolite of the opioid buprenorphine that is formed by glucuronidation of buprenorphine by a UGT.

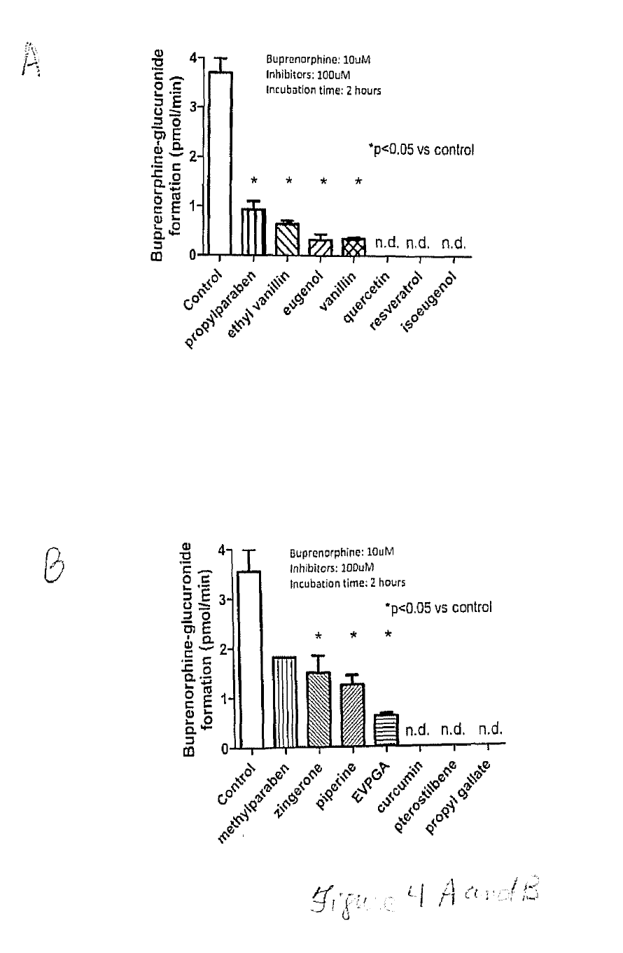

FIG. 4A shows that propylparaben, ethyl vanillin, eugenol, and vanillin each significantly decreased the formation of buprenorphine glucuronide; the formation of buprenorphine glucuronide was not quantitated for quercetin, resveratrol, or isoeugenol since it was not detected (n.d.). FIG. 4B shows that zingerone, piperine, and ethyl vanillin propylene glycol acetal (EVPGA) each significantly decreased the formation of buprenorphine glucuronide; the formation of buprenorphine glucuronide was not quantitated for curcumin, pterostilbene, or propyl gallate since it was not detected (n.d.). One determination with methylparaben was available and showed a trend toward a decrease in buprenorphine glucuronidation. In conclusion, propylparaben, ethyl vanillin, eugenol, vanillin, quercetin, resveratrol, isoeugenol, zingerone, piperine, ethyl vanillin propylene glycol acetal (EVPGA), curcumin, pterostilbene, propyl gallate, and methylparaben decreased the formation of buprenorphine glucuronide.

### Example 2

### Effects of Other Inhibitors the Formation of Buprenorphine-Glucuronide in LS 180 Cells

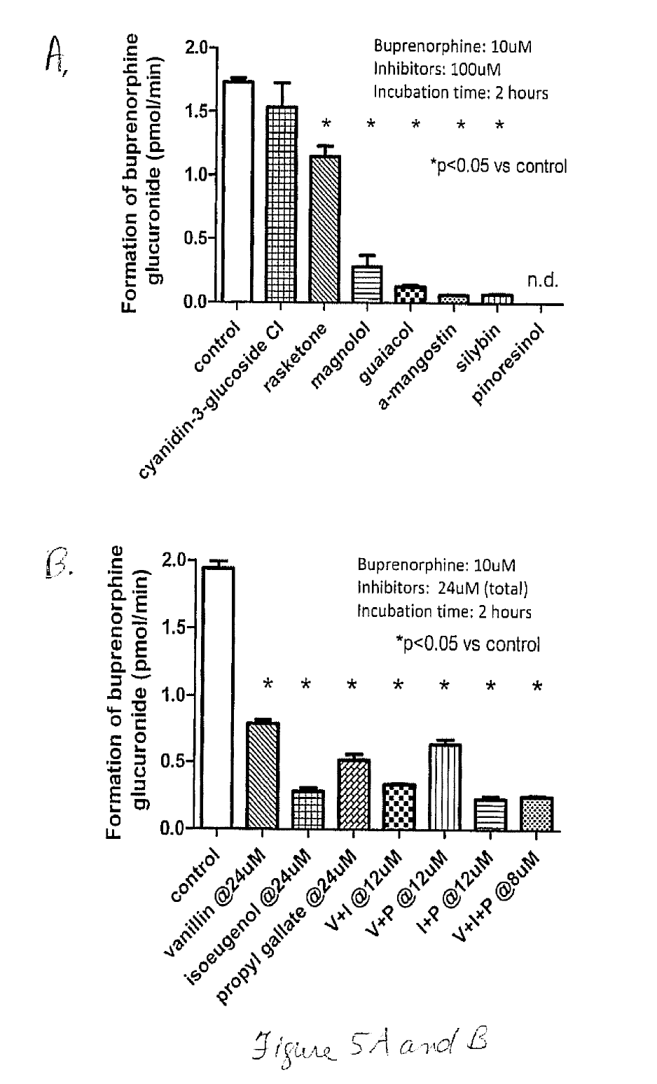

Cells were cultured, treated, and samples analyzed as described for Example 1 with buprenorphine (10 μM) in the presence or absence of inhibitors, as listed (100 μM) in FIG. 5A and B. FIG. 5A shows that rasketone, magnolol, guaiacol, α-mangostin, and silybin each significantly decreased the formation of buprenorphine glucuronide; the formation of buprenorphine glucuronide was not quantitated for pinoresinol since it was not detected (n.d.). In contrast, cyanidin-3-glucoside did not have a significant effect. These results show that rasketone, magnolol, guaiacol, α-mangostin, silybin and pinoresinol decreased the formation of buprenorphine glucuronide.

### Example 3

### Synergy of Inhibitor Combinations

Cells were cultured, treated, and samples analyzed as described above (Example 1) with buprenorphine (10 μM) in the presence or absence of the following treatments: vanillin (24 μM), isoeugenol (24 μM), propyl gallate (24 μM), vanillin and isoeugenol (12 μM each, together), vanillin and propyl gallate (12 μM each, together), isoeugenol and propyl gallate (12 μM each, together), and a combination of vanillin, isoeugenol, and propyl gallate (8 μM each, all together). The results (FIG. 5B) show that each of these treatments significantly inhibited the formation of buprenorphine glucuronide (p<0.05). Although the combination of vanillin & propyl gallate was merely additive (yielding an effect equal to the average of both inhibitors), other combinations (isoeugenol and propyl gallate; vanillin & isoeugenol; vanillin & isoeugenol & propyl gallate) were synergistic (yielding an effect greater than the average of the inhibitors). These results indicate that certain combinations of inhibitors yield an unexpected synergistic effect on inhibiting the formation of buprenorphine glucuronide.

The partition coefficient is a ratio of concentrations of un-ionized compound between the two solutions. To measure the partition coefficient of ionizable solutes, the pH of the aqueous phase is adjusted such that the predominant form of the compound is un-ionized. The logarithm of the ratio of the concentrations of the un-ionized solute in the solvents is called log P: The log P value is also known as a measure of lipophilicity.

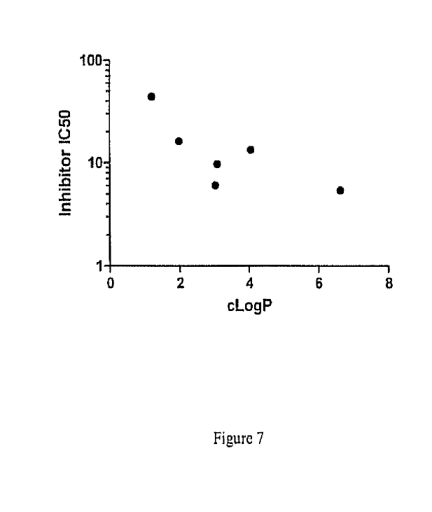

FIG. 7 shows a plot of the IC₅₀ values for the inhibitors isoeugenol, pterostilbene, quercetin, resveratrol, vanillin, and a-mangostin vs the calculated Log P values (c Log P; ACS SciFinder). The plot demonstrates that c Log P is only loosely related to the IC₅₀ values of the inhibitors, therefore, simple lipophilicity is not sufficient to predict the extent to which these compounds inhibit buprenorphine glucuronidation.

### Example 4

### Phenolic Opioids: Tests Conducted in vivo

Healthy jugular-vein cannulated male rats (280-350 g) were dosed by oral gavage with buprenorphine 10 mg/kg, naloxone 2.5 mg/kg with or without an inhibitor combination. The liquid formulation was an aqueous spontaneous micro/nanoemulsion formulation containing 0.94% w/v polyethylene glycol 400 (PEG400), 0.25% w/v polysorbate 80 (TWEEN® 80), and 0.49% tocopherol polyethylene glycol (1000) succinate (TPGS). The inhibitor combination comprised a near-eutectic mixture of eugenol 20 mg/kg, isoeugenol 16 mg/kg, ethyl vanillin 20 mg/kg, vanillin 20 mg/kg, curcumin 5 mg/kg, silybin A 5 mg/kg, α-mangostin 5 mg/kg, resveratrol 20 mg/kg, propyl gallate 12 mg/kg, and naringin 60 mg/kg.

Following dosing, plasma samples (100 μL) were obtained at 2 hours after dosing. Samples were spiked with pentazocine (as an internal standard), alkalinized with 10 μL of ammonium bicarbonate (1M, pH 9.3), extracted into ethyl acetate:hexane 4:1 (400 μL twice), evaporated under reduced pressure, and reconstituted with 100 μL mobile phase comprised of 70% acetonitrile 30% aqueous (0.2% acetic acid, 0.2% triethylamine, ammonium hydroxide qs to pH 6.0). Buprenorphine alone was separated by HPLC using an Alltech Alltima HP C18 3 μm 4.6×100 mm column and quantitated by fluorescence detection (excitation 214 nm, emission 352 nm) with pentazocine as an internal standard (excitation 210 nm, emission 346 nm). The calibration range was from 7.8 to 2000 nM.

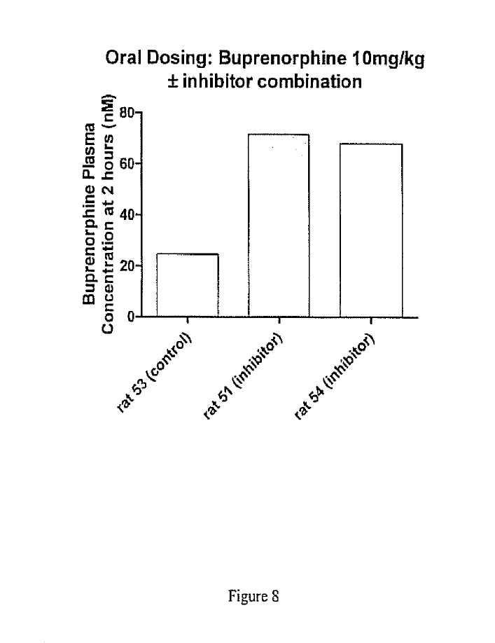

The results are presented in FIG. 8. The results demonstrate a >3-fold increase in the plasma concentrations at two hours after oral dosing. This time point is expected to be near the peak of the concentration-time profile.

These results show that a combination of inhibitors when given with buprenorphine increases the plasma concentrations (and hence, bioavailability) of buprenorphine.

### Example 5

### Exemplary Oral Formulation

| | |
|---|---|
| buprenorphine HCl: | 24 mg |
| naloxone HCl: | 6 mg |
| polysorbate (Tween) 80: | 20 mg |
| D-α-tocopheryl polyethylene glycol 1000 succinate (TPGS): | 50 mg |
| inhibitor combination (as described below): | 300 mg |
| polyethylene glycol (PEG) 400: | 200 mg |

TOTAL: 600 mg in a size 1 or size 0 gel- or liquid-filled capsule

The inhibitors form a eutectic or partially-eutectic mixture, in that they tend to liquify upon mixing, thus enabling their formulation in a gel or liquid-filled capsule. For fully-solid dosage forms (such as tablets), higher molecular weight polyethylene glycol (e.g. 4000) is substituted for those of lower molecular weights (e.g. 400), or replaced with diluents such as silicic acid, magnesium oxide, or other adsorbents.

An exemplary inhibitor combination is comprised of resveratrol 100 mg, curcumin 50 mg, quercetin 50 mg, isoeugenol 50 mg, and propyl gallate 50 mg. Other inhibitor combinations as described above may also be used.

All publications, patent applications and issued patents cited herein are hereby incorporated by reference in entirety.

While the invention has been described in terms of several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of providing one or more opioids to a subject in need thereof so as to enhance systemic bioavailiability of said one or more opioids, comprising the step of
providing said one or more opioids to said subject in combination with one or more inhibitors of one or more uridine diphosphate glucuronosyl transferases (UGTs), wherein said one or more inhibitors of one or more UGTs are classified as Generally Regarded as Safe (GRAS), “Everything Added to Food” (EAF) and/or as a dietary supplement.

2. The method of claim 1, wherein said one or more opioids includes buprenorphine.

3. The method of claim 1, wherein said one or more opioids includes buprenorphine and naloxone.

4. The method of claim 1, wherein said one or more inhibitors of one or more UGTs inhibits at least one of UGT1A1, UGT1A3, UGT1A4, UGT1A5, UGT1A6, UGT1A7, UGT1A8, UGT1A9, UGT1A10, UGT2A1, UGT2A2, UGT2A3, UGT2B4, UGT2B7, UGT2B10, UGT2B11, UGT2B15, UGT2B17 and UGT2B28.

5. The method of claim 1, further comprising providing said subject with one or both of:
i) one or more inhibitors of at least one cytochrome P450 monooxygenase (CYP) selected from the group consisting of CYP1A1, CYP1A2, CYP2A6, CYP2D6, CYP2C9, CYP2C8, CYP2C18, CYP2C19, CYP3A4, CYP3A5 and CYP3A7; and
ii) one or more inhibitors of at least one sulfotransferase (SULT) selected from the group consisting of SULT1A1, SULT1A2, SULT1A3, SULT1C4 and SULT2B1.

6. The method of claim 1, where said one or more inhibitors is selected from the group consisting of propylparaben, ethyl vanillin, eugenol, vanillin, quercetin, resveratrol, isoeugenol, methylparaben, zingerone, piperine, ethyl vanillin propylene, glycol acetal, curcumin, pterostilbene, propylgallate, rasketone, magnolol, guaiacol, a-mangostin, silybin, and pinoresinol.

7. The method of claim 6 wherein said one or more inhibitors is administered as a combination of inhibitors selected from the group consisting of: isoeugenol and propyl gallate; vanillin and isoeugenol; and vanillin, isoeugenol and propyl gallate.

8. The method of claim 1, wherein said step of providing is performed orally or intra-rectally.

9. A dosage form of one or more opioids, comprising
one or more opioids and
one or more UGT inhibitors that is classified as Generally Regarded as Safe (GRAS), “Everything Added to Food” (EAF) and/or as a dietary supplement.

10. The dosage form of claim 9, wherein said one or more opioids includes buprenorphine.

11. The dosage form of claim 10, wherein said one or more opioids include buprenorphine and naloxone.

12. The dosage form of claim 10, wherein said one or more inhibitors of one or more UGTs inhibits at least one of UGT1A1, UGT1A3, UGT1A4, UGT1A5, UGT1A6, UGT1A7, UGT1A8, UGT1A9, UGT1A 10, UGT2A1, UGT2A2, UGT2A3, UGT2B4, UGT2B7, UGT2B10, UGT2B11, UGT2B15, UGT2B17 and UGT2B28.

13. The dosage form of claim 12, further comprising one or both of:
i) one or more inhibitors of at least one cytochrome P450 monooxygenase (CYP) selected from the group consisting of CYP1A1, CYP1A2, CYP2A6, CYP2D6, CYP2C9, CYP2C8, CYP2C18, CYP2C19, CYP3A4, CYP3A5 and CYP3A7; and
ii) one or more inhibitors of at least one sulfotransferase (SULT) selected from the group consisting of SULT1A1, SULT1A2, SULT1A3, SULT1C4 and SULT2B1.

14. The dosage form of claim 9, where said one or more inhibitors is selected from the group consisting of propylparaben, ethyl vanillin, eugenol, vanillin, quercetin, resveratrol, isoeugenol, methylparaben, zingerone, piperine, ethyl vanillin propylene, glycol acetal, curcumin, pterostilbene, propyl gallate, rasketone, magnolol, guaiacol, a-mangostin, silybin, and pinoresinol.

15. The dosage form of claim 14, wherein said one or more inhibitors is administered as a combination of inhibitors selected from the group consisting of: isoeugenol and propyl gallate; vanillin and isoeugenol; and vanillin, isoeugenol and propyl gallate.

16. The dosage form of claim 9, wherein said dosage form is formulated for oral, rectal, vaginal or urethral administration.

17. A method of treating or preventing pain in a subject in need thereof, comprising
administering to said subject a composition comprising
one or more opioids and
one or more UGT inhibitors that is classified as Generally Regarded as Safe (GRAS), “Everything Added to Food” (EAF) and/or as a dietary supplement.

18. The method of claim 17, wherein said one or more opioids includes buprenorphine.

19. The method of claim 17, wherein said one or more UGT inhibitors is a combination of inhibitors selected from the group consisting of: isoeugenol and propyl gallate; vanillin and isoeugenol; and vanillin, isoeugenol and propyl gallate.

20. The method of claim 1, wherein the one or more inhibitors is a combination of at least two inhibitors selected from the group consisting of eugenol, isoeugenol, ethyl vanillin, vanillin, curcumin, silybin A, a-mangostin, resveratrol, propyl gallate, and naringin.

21. A method of treating or preventing opiate dependency or addiction in a subject in need thereof, comprising
administering to said subject a composition comprising
buprenorphine and naloxone; and
one or more UGT inhibitors that is classified as Generally Regarded as Safe (GRAS), “Everything Added to Food” (EAF) and/or as a dietary supplement.

22. The method of claim 21, wherein said one or more UGT inhibitors are selected from the group consisting of eugenol, isoeugenol, ethyl vanillin, vanillin, curcumin, silybin A, a-mangostin, resveratrol, propyl gallate, and naringin.

23. The method of claim 21, wherein said one or more UGT inhibitors includes 2, 3, 4, 5, 6, 7, 8, 9 or 10 inhibitors.

24. The method of claim 21, wherein said one or more UGT inhibitors is a combination of inhibitors selected from the group consisting of: isoeugenol and propyl gallate; vanillin and isoeugenol; and vanillin, isoeugenol and propyl gallate.

25. The method of claim 1, wherein said one or more UGT inhibitors includes a combination of alkylated catechols and other phenolic compounds.

26. The method of claim 17, wherein the one or more inhibitors is a combination of at least two inhibitors selected from the group consisting of eugenol, isoeugenol, ethyl vanillin, vanillin, curcumin, silybin A, a-mangostin, resveratrol, propyl gallate, and naringin.

27. The oral dosage form of claim 9, wherein the one or more inhibitors is a combination of at least two inhibitors selected from the group consisting of eugenol, isoeugenol, ethyl vanillin, vanillin, curcumin, silybin A, a-mangostin, resveratrol, propyl gallate, and naringin.

28. The method of claim 17, wherein said one or more UGT inhibitors includes a combination of alkylated catechols and other phenolic compounds.

29. The method of claim 21, wherein said one or more UGT inhibitors includes a combination of alkylated catechols and other phenolic compounds.

30. The oral dosage form of claim 9, wherein said one or more UGT inhibitors includes a combination of alkylated catechols and other phenolic compounds.
